(19) United States
(12) Patent Application Publication
(10) Pub. No.: US 2016/0106664 A1
(43) Pub. Date: Apr. 21, 2016

(54) NOVEL PROPELLANT CONTAINING PREPARATIONS FOR TIOTROPIUM

(71) Applicant: Boehringer Ingelheim International GmbH, Ingelheim am Rhein (DE)

(72) Inventors: Bettina Berner, Ingelheim am Rhein (DE); Hubert Hoelz, Oberheimbach (DE); Mariola Mann, Bingen am Rhein (DE)

(21) Appl. No.: 14/982,263

(22) Filed: Dec. 29, 2015

Related U.S. Application Data
(63) Continuation of application No. 14/727,947, filed on Jun. 2, 2015, now abandoned.
Application No. 14/727,947 is a continuation of application No. 13/779,909, filed on Feb. 28, 2013, now abandoned.

(30) Foreign Application Priority Data
Feb. 28, 2012 (EP) 12 157 237.4

(51) Int. Cl. A61K 9/00; A61M 16/20; A61M 15/00; A61K 9/12; A61K 31/439

(52) U.S. Cl. CPC A61K 9/008; A61K 9/124; A61K 31/439; A61M 15/009; A61M 16/20

(57) ABSTRACT

The present invention relates to a novel propellant-gas-containing medicament composition based on tiotropium 1, processes for the preparation thereof and the use of such medicament compositions in the treatment of respiratory complaints.

The invention relates to pressurised gas preparations for metered dose aerosols in which the active substance tiotropium bromide is formulated in solution in HFA-propellant gases (hydrofluoroalkanes) as propellant, and the use thereof for preparing a medicament, as well as the preparation of pressurised gas preparations of this kind. Preferably it relates to an inhalable aerosol.

### BACKGROUND

In the treatment of diseases there is a need for medicaments which are suitable, by virtue of their special metering, on the one hand for treating a disease (primary therapeutic objective) and on the other hand for keeping potential side effects to a minimum. Thus by the provision of special metering the intention is to achieve medical treatment based on a patient-oriented decision, if possible on the basis of empirically determined efficacy, while taking account of potential side effects. Consequently, the aim of such a treatment is to achieve the desired effect with the minimum side effects. In particular, such an approach means that particular drug therapies have to be provided for specific groups of patients (patient-oriented, individualised therapy).

In order to be able to monitor such drug treatments within the scope of medicaments for administration by inhalation, apart from the medical diagnostic category and individual assessment of a patient, it is necessary to provide precisely determined doses of the medicament in order to achieve both an “optimum efficacy” and also a “minimum of side effects” in the treatment of a patient. For this objective the dosage of an inhaled medicament has to be defined using numerous dosage parameters (preferably in parallel, using several dosage parameters).

Lung diseases are usually treated by topical application of an active substance to the surface of the lungs. Using inhalative therapies of this kind means that tiny amounts of active substance are distributed as uniformly as possible over the surface of the lungs by the patient breathing them in. Thus, the treatment of lung diseases carried out by topical drug therapy using an aerosol for inhalation, places special demands on the dosage accuracy of a topical administration of active substance by inhalation.

For treating lung diseases (e.g. COPD and asthma), particularly for Chronic Obstructive Pulmonary Diseases, the active substance tiotropium 1

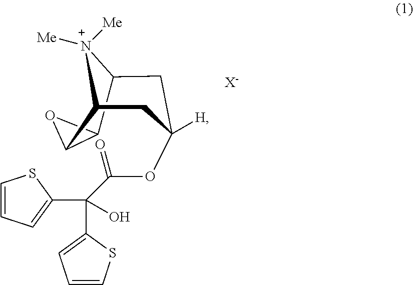

wherein X⁻ denotes a negatively charged anion, preferably selected from among chloride, bromide, iodide, sulphate, phosphate, methanesulphonate, nitrate, maleate, acetate, citrate, fumarate, tartrate, oxalate, succinate, benzoate and p-toluenesulphonate, particularly bromide (EP0418716), is used.

Because of the high potency of tiotropium, the topical drug treatment of lung diseases with tiotropium means that a daily dose (in vivo) of a few μg is sufficient to achieve an effective treatment of COPD. In such a treatment, about 0.5 μg to 4 μg of substance are administered to the surface of the lungs by inhalation of the active substance. Such administration may be carried out for example using the product known as SPIRIVA®/Handihaler®. With an estimated lung surface area of 70 m² and an average geometric particle size of 8 μm³ (further computed assumptions: density of 3 g/cm³, lung dosage 3 μg) this is computed to correspond to the distribution of 1 microparticle per 5-6 cm² of lung surface area.

As only a fraction of the nominal dose consequently reaches the target organ of the “lung surface” when a medicament is administered by inhalation, there is therefore only a very slight coating of the lung surface (particles per unit of lung surface) with the active substance, but this is still sufficient to achieve a therapeutic effect.

The difference (hereinafter the sum of X₂ₐ and X_{2b}) between the nominal dose Y and the pulmonary dose X₁ is not therapeutically relevant in the administration of an active substance by inhalation, in that the desired therapeutic effect is achieved provided that the pulmonary dose X₁ is within the desired range.

However, the difference between the nominal dose and the pulmonary dose is relevant in terms of whether
(i) the therapy is economical or uneconomical, and/or even whether
(ii) an increased rate of side effects is to be expected.

This difference between the nominal dose Y and the pulmonary dose X₁ in turn consists of a partial amount which is left behind in the applicator device for technical reasons during the delivery (partial amount X₂ₐ) and is consequently not supplied to the patient, and a second partial amount which the patient takes up on inhaling the product but which is not delivered to the target organ of the “lung surface” and therefore does not reach the target organ of the “lung surface” (partial amount X_{2b}). For example, this is the proportion of active substance that is deposited in the oro-pharyngeal space and is then swallowed by the patient.

It should also be pointed out that this partial amount X_{2b} in particular is responsible for the side effect profile, as the active substance tiotropium, which should be available to/act on the “lung surface” solely topically from a therapeutic point of view, is made available to the body by another method, depending on the actual magnitude of the partial amount X_{2b}. This active substance fraction X_{2b} of the dose means that unwanted effects of the active substance may be observed in the body (side effects).

To summarise, for the administration by inhalation of an active substance the nominal dose Y supplied by administration, i.e. by the process of inhalation by the patient, has to be broken down functionally into a pulmonary dose X₁, a partial dose which is not available to the patient (wasted fraction) X₂ₐ, which is left behind in the inhaler, for example, and a partial amount of the dose which the patient takes but which does not reach the target organ of the “lung surface” X_{2b}, in order to describe the dose of a medicament administered by inhalation. An optimum ratio between the partial amounts of the dose of active substance administered brings about an optimisation of the activity and side effect profile of the medicament.

The following equation applies to the dose:

Y=X₁+(X₂ₐ+X_{2b})

Inhalative administration of an active substance is possible in principle by various pharmaceutical techniques. Techniques for the inhalative administration of an active substance encompass the possibilities of the active substance being administered for example as a powder formulation (WO 02/30389) or alternatively made available to the patient as a formulation in the form of a solution, the administration being carried out with a nebuliser (Respimat®portable device) (WO 03/084519).

Another form of inhalative administration known to the skilled man is the administration of an inhaled dose by means of a pressurised-gas-operated metered dose aerosol. In this, a formulation in which the active substance is present in dissolved or suspended form is divided into portions by the admixture of a liquefied propellant gas by means of a metering valve, and as a result of its expulsion from the nozzle head and the consequent relaxation of pressure it is converted into an inhalable aerosol (WO 03/000241, EP2322243, WO2006/002840).

Each of these special inhalative preparations requires special formulations for the particular method of administration (e.g. powder inhalation, Soft-Mist-Inhaler Respimat®, propellant-gas-operated metered dose aerosol). From a pharmaceutical-technical and medical point of view the objective is to optimise the product or formulation for the chosen method of delivery so that for a pulmonary dose X₁ that is desirable from a medical point of view an optimised ratio of the magnitudes X₁ to Y, X₁ to X₂ₐ or X₁ to X_{2b} is achieved. A particular aim is to optimise the ratio of X₁ to X_{2b}.

In particular the present invention sets out to provide inhalable aerosol formulations for the active substance tiotropium in which the active substance is dissolved and can thus be administered by a propellant-gas-operated metering aerosol with a high degree of metering accuracy, while in particular the metering accuracy is maintained over the period of operation and consequently the stability of the inhalation solution as a function of time is also guaranteed.

### DEFINITIONS

### Dosage Parameter “Nominal Dose Y”:

By the nominal dose Y is meant the metered dose. The nominal dose corresponds to the quantity of active substance that is theoretically to be provided to the patient by administration using the inhaler for each single actuation of the latter. The nominal dose is hence the quantity of active substance that can be determined by computation from the magnitudes active substance concentration and metering chamber volume.

### Dosage Parameter “Pulmonary Dose X₁”:

By the pulmonary dose X₁ is meant the quantity of active substance that is available for inhalation when the active substance is delivered as a lung-bound aerosol. The pulmonary dose X₁ corresponds to the Fine Particle Dose as defined in the pharmacopoeias. According to the present invention the pulmonary dose is determined as the Fine Particle Dose (Pharm. Eur. 2.9.18, European Pharmacopeia, 6th Edition 2008, Apparatus D, Andersen Cascade Impacter) or USP30-NE25 <601>).

### Dosage Parameter “Delivered Dose DD”:

The delivered dose (DD) is the dose that is theoretically delivered to the patient by the inhaler. It is determined according to the Pharmacopoeia (Pharm Eur 0671, USP <601> Delivered Dose Apparatus A) on the basis of the measurement with a delivery unit. The dosage collecting tube comprises, for the measurement, an adapter tailored to the device used (mouthpiece).

Dosage Parameter “Partial Dose which is not Available to the Patient X₂ₐ”:

According to the invention the dose X₂ₐ corresponds to the nominal dose Y minus the delivered dose DD.

The equation is:

X₂ₐ=Y−DD

Dosage Parameter “Partial Amount of the Dose which is Taken by the Patient but which does not Reach the Target Organ of the “Lung Surface” X_{2b}”:

The partial dose X_{2b} which the patient takes but which does not reach the target organ of the “lung surface” corresponds to the delivered dose DD minus the pulmonary dose X₁.

The equation is:

X_{2b}=DD−X₁

### Dosage Parameter “Mean Aerodynamic Particle Size MMAD”:

According to the invention the term mean aerodynamic particle size MMAD denotes the aerodynamically mean mass diameter. This variable is determined according to the Pharmacopoeia (Pharm Eur 2.9.18 Apparatus D, USP USP <601> Aerodynamic size distribution Apparatus 1) based on measurement with an Anderson cascade impactor. Depending on the aerodynamic particle size the particles are deposited in the lungs at different depths (e.g. in the bronchi, the bronchioles, or the alveoli, etc.). The selected aerodynamic particle size thus serves to control where, in the lungs, the active substance will preferably settle and thus be available.

### “Lung Surface”:

The lung surface denotes the inner surface area of the airways in the lungs. It amounts to about 70 m².

### Solution-Type Metered Dose Aerosol:

A metered-dose aerosol is a preparation for active substances which are intended for inhalation by the patient. In a metered-dose aerosol the active substance is released in measured doses as an aerosol, i.e. as finely divided droplets of liquid in a gas phase. The release of a single dose is referred to as a spray jet. Solution-type metered dose aerosols contain the active substance in dissolved form in the propellant gas and a cosolvent, the active substance solution being nebulised by means of propellant gas. The active substance solution is located in a medicament container (cartridge) with mouth tube, this container being filled with propellant gas, and finely divided active substance is released in a measured dose when pressure is applied.

### Activity/Side Effects Profile:

A side effect is an effect of a medicament which occurs alongside the intended main effect but which does not constitute the actual treatment goal (main effect) of the medicament in the sense of the present invention (an unwanted side effect is hereinafter equated with the term “side effect”). Such a side effect may optionally occur within the scope of the administration of a medicament. By the effect of a medicament is meant the therapeutically intended main effect of a medicament. Side effects can be divided into (i) drug-typical and dosage-dependent side effects and (ii) dosage-independent side effects (e.g. hypersensitivity reactions). For medicaments administered by inhalation both the effect and dosage-dependent side effects (i) are dependent on the properties/the nature of the formulation and the particular method of administration (e.g. metered-dose aerosol, powder inhalation, etc.). Consequently, the effect and dosage-dependent side effect are observed as a function of the specifically selected combination of the nominal dose Y, the pulmonary dose X₁ and the delivered dose DD, and the variables dose X₂ₐ, dose X_{2b} and the aerodynamic particle size (especially the MMAD) and the method of administration selected.

### Determination of Tiotropium Bromide Content in the Formulation:

The content of tiotropium 1 in the form of tiotropium bromide can be determined by HPLC on the basis of an isocratic method.

### Parameters of the Method:

Column: ACE 3 C18, 75 mm×4.6 mm (manufacturer: ACE®)

### Detection: UV, 235 nm

Eluant: 70% KH₂PO₄ buffer, pH 3.0/30% methanol
Sample volume: 10 μl
Flow: 1.5 ml/min

### Determination of pH:

By the pH of the propellant-containing solution formulations is meant the corresponding pH which can be obtained from the measured value by conversion according to the reference “Interpretation of pH Measurements in Alcohol-Water Solvents”, J. Phys. Chem. Volume 67, September 1963, pages 1833-1838. The measured value that is used in the conversion can be obtained directly using a pH electrode from the ethanolic solution, or the solution of the active substance in the cosolvent(s).

### Method of Measuring the pH:

pH-electrode: For pH measurements glass electrodes (KCl electrode) with bridge electrolyte are used. These pH electrodes are filled at the factory with 3 molar KCl solution as the bridge electrolyte, as standard, and can be used directly.

Procedure: The measurement is carried out at ambient temperature (23° C.+/−2° C.). Unless stated otherwise by the manufacturer of the pH electrode, first the “zero point” [mV] of the electrode is determined with buffer solution pH 7.

In non-automatic devices, the pH of the buffer solution is adjusted using the pH meter as a function of the temperature and the associated voltage [mV] and temperature [° C.] are read off. To determine the “steepness” a second buffer is used, the value of which differs from pH 7 by at least 2 pH units and which encompasses the pH of the sample that is to be measured. The measurement is carried out as specified in the operating instructions provided by the manufacturer of the pH meter/pH electrode.

Before changing to a new sample, the electrode should be rinsed with deionised water and residual drops should be carefully wiped off.

The measured value (pH and parallel measurement of the temperature) is read off as soon as the value remains constant (±0.05 pH units) (at least 20 seconds).

### Normality (“e.g. 0.01 Normal Acid”):

The expression of the normality (in units: N) gives the equivalent concentration c_{eq} that constitutes a typical concentration in chemistry. It is a special concentration of substances based on a reference factor of 1/z. z is the stoichiometric valency, also known as the equivalent number. Where z=2 the equivalent concentration is thus twice as great as the concentration of substances, as each whole particle is counted z times, so to speak. The term 1/z is also referred to as equivalent particles or equivalents. C_{eq} is a measurement of how many equivalent particles of a substance are present in a specified volume of the solution.

The normality is then defined by the equation:

N=nᵥₐₗ/V.

Here, N is the normality, nᵥₐₗ is the quantity of equivalents and V is the volume.

For example, in the case of sulphuric acid, two protons may attach to a sulphate ion (SO₄²⁻), corresponding to the valency of the acid ion. Consequently, the solution contains twice as many equivalent particles (in this case protons) as molecules of the substance itself.

H₂SO₄→SO₄²⁻+H⁺,

i.e. 1 mol/l (H₂SO₄)=2N (H₂SO₄) or in other words a 1 normal H₂SO₄ solution is ½ molar (1N in this example corresponds to ½ M).

### BRIEF DESCRIPTION OF SEVERAL VIEWS OF THE DRAWING

FIG. 1: Inhaler containing medicinal solution, where (a) depicts a cartridge containing a propellant gas-containing solution, and (b) depicts a nozzle characterized by a bore diameter.

FIG. 2: An expanded view of the Inhaler containing medicinal solution, where (c) depicts the metering chamber of the cartridge.

### DESCRIPTION OF THE INVENTION

The present invention relates to a novel propellant-gas-containing medicament composition based on tiotropium 1, processes for the preparation thereof and the use of such medicament compositions in the treatment of respiratory diseases.

Surprisingly, an unexpected, beneficial therapeutic effect can be demonstrated, particularly taking account of a reduced rate of side effects in the treatment of inflammatory or obstructive respiratory diseases and/or asthma, if tiotropium bromide is used as a solution-type metered dose aerosol in a specific dosage. On the basis of this special dosage, the tiotropium-containing medicament compositions according to the invention can also be used in particular as a monotherapy, while the activity/side effect profile is optimised by the particular dosage to be used. Medicament formulations of this kind are characterised according to the invention by an improved pharmaceutical stability (chemical and physical), particularly also by an optimum stability while at the same time keeping the above dosage requirements over time.

Within the scope of the present patent application, the term tiotropium 1 denotes salts. In the above-mentioned salts the tiotropium cation represents the pharmacologically active ingredient. Within the scope of the present patent application an explicit reference to the above cations is indicated by the use of the number 1′. By the salts 1 which may be used within the scope of the present invention are meant the compounds which contain, in addition to tiotropium, as counter-ion (anion), chloride, bromide, iodide, sulphate, methanesulphonate or para-toluenesulphonate. Within the scope of the present invention, the methanesulphonate, chloride, bromide and iodide are preferred of all the salts 1, the methanesulphonate and bromide being of particular importance. Of outstanding importance according to the invention are salts 1 selected from among tiotropium bromide, tiotropium chloride and tiotropium methanesulphonate. Tiotropium bromide is particularly preferred.

The substance 1 is administered by producing an inhalable aerosol, by spraying a pressurised active substance solution. Inhalable aerosols of this kind contain a propellant gas to produce the inhalable aerosol. The propellant gases that can be used to prepare inhalable aerosols are known from the prior art. The propellant-gas-containing inhalable aerosols according to the invention contain the propellant gas HFA 134a. In another embodiment, the propellant-gas-containing inhalable aerosols according to the invention may contain, in addition to the propellant gas HFA 134a, other propellant gases which are selected for example from among the hydrocarbons such as n-propane, n-butane or isobutane and halohydrocarbons such as fluorinated derivatives of methane, ethane, propane, butane, cyclopropane or cyclobutane. The above-mentioned propellant gases may be used in addition to the propellant gas HFA 134a on their own or in mixtures together with the propellant gas HFA 134a.

Particularly preferred according to the invention are propellant-gas-containing inhalable aerosols which contain only HFA134a as propellant gas. The propellant-gas-containing inhalable aerosols according to the invention may also contain other ingredients such as cosolvents, stabilisers, surfactants, antioxidants, lubricants and preservatives. All these other ingredients are known in the art.

The formulations according to the invention for preparing propellant-gas-containing inhalable aerosols contain means for adjusting the pH. The propellant-gas-containing inhalable aerosols according to the invention have a pH of 6-8, preferably 6.5-7.5. The pH of the propellant-gas-containing inhalable aerosols corresponds according to the invention to the pH that can be obtained by conversion from the measured value. The measured value used for the conversion corresponds to the value that can be measured by a pH electrode directly in the solution of the active substance in the cosolvent(s), preferably in the solution of active substance and ethanol and water. The propellant-gas-containing inhalable aerosols according to the invention contain an acid, preferably selected from among inorganic or organic acids.

Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid. Preferred inorganic acids are hydrochloric and sulphuric acids. It is also possible to use the acids which already form an acid addition salt with the active substance. Of the organic acids, ascorbic acid, fumaric acid and citric acid are preferred. If desired, mixtures of the above acids may be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH. In another embodiment according to the invention citric acid is particularly preferably used. Preferably, propellant-gas-containing inhalable aerosols contain citric acid in an amount of 0.005-0.1%, particularly preferably in an amount of 0.05-0.1%, particularly preferably 0.04-0.09%, most particularly preferably 0.03-0.08%, also particularly preferably 0.02-0.05%.

In another preferred embodiment the propellant-gas-containing inhalable aerosols according to the invention contain an inorganic acid in a concentration that corresponds to a quantity of H⁺-ions which can be obtained by adding a 1-3% proportion of a 0.01 normal acid, preferably an inorganic acid in a concentration that corresponds to a quantity of H⁺-ions which can be obtained by adding a 1.5-2.5% proportion of a 0.01 normal acid.

In another preferred embodiment the propellant-gas-containing inhalable aerosols according to the invention contain hydrochloric acid and/or hydrobromic acid, preferably hydrochloric acid, as the inorganic acid.

In another preferred embodiment the propellant-gas-containing inhalable aerosols according to the invention contain sulphuric acid as the inorganic acid.

In another preferred embodiment the propellant-gas-containing inhalable aerosols according to the invention contain an organic acid selected from among ascorbic acid, fumaric acid and citric acid.

In another preferred embodiment the propellant-gas-containing inhalable aerosols according to the invention contain ascorbic acid as the inorganic acid.

The proportion of dissolved pharmaceutically active substance 1 in the finished preparation (based on a metering chamber of 50 μl) is 0.005-0.05% according to the invention. Preferably, the formulations according to the invention for preparing propellant-gas-containing inhalable aerosols contain 0.005-0.03% of the dissolved active substance 1. Also preferably, the formulations according to the invention for preparing propellant-gas-containing inhalable aerosols contain 0.008-0.045% of the dissolved active substance 1. Also preferably, the formulations according to the invention for preparing propellant-gas-containing inhalable aerosols contain 0.01-0.04% of the dissolved active substance 1. Also preferably, the formulations according to the invention for preparing propellant-gas-containing inhalable aerosols contain 0.01-0.02% of the dissolved active substance 1. Also preferably, the formulations according to the invention for preparing propellant-gas-containing inhalable aerosols contain 0.02% of the dissolved active substance 1.

The overall composition according to the invention of the propellant-gas-containing inhalable aerosols is obtained from the percentage of dissolved active substance (% by weight) plus the percentage of cosolvents (% by weight), plus the percentage of acid (% by weight), the remainder up to 100% being made up by the propellant gas. If the inhalable aerosols according to the invention contain other ingredients such as stabilisers, surfactants, antioxidants, lubricants and preservatives, these are present in an amount of less than 2% (% by weight), preferably less than 1%, while the amount of propellant gas is reduced accordingly by the presence of these additional components. In one particular embodiment, the inhalable aerosols according to the invention contain only active substance, cosolvents, acid and propellant gas.

The medicament formulations according to the invention are administered as single doses by means of a spray jet, in which a dose flows out of the metering chamber by actuation of the inhaler by expulsion from the nozzle. The metering chamber has a capacity of between 25 μl and 100 μl, preferably 50 μl. According to the invention the details of the composition of the medicament formulation (proportion of the acid in % by weight and proportion of the dissolved active substance 1 in % by weight) for determining the dose are based on a metering chamber with a volume of 50 μl, unless stated otherwise. By suitable calculation adjustments (e.g. doubling, halving) the skilled man can also obtain the corresponding concentrations for the proportions of acid and active substance for a metering chamber of for example 25 μl and 100 μl and these are encompassed by the invention.

As another embodiment according to the invention the metering is carried out by 2 spray jets immediately following one another, preferably within less than 10 minutes, so that the overall dose corresponds to the administration of 2 partial doses of 1 spray jet. In this embodiment of the invention the nominal dose and the further dosage details according to the invention that can be derived from it relate to the total amount administered in the two spray jets.

This metering is particularly suitable for treating a medium- or high-grade COPD.

Medicament formulations according to the invention which are administered as single doses, the metering chamber having a volume of 25 μl, preferably contain the dissolved medicament 1 in an amount of 0.01-0.1%, particularly preferably in an amount of 0.01-0.09%, particularly preferably in an amount of 0.016-0.09%, particularly preferably in an amount of 0.02-0.08%, particularly preferably in an amount of 0.02-0.04%, particularly preferably in an amount of 0.04%. Medicament formulations according to the invention which are administered as single doses, the metering chamber having a volume of 50 μl, preferably contain the dissolved medicament 1 in an amount of 0.005-0.05%, particularly preferably in an amount of 0.005-0.045%, particularly preferably in an amount of 0.008-0.04%, particularly preferably in an amount of 0.01-0.02%, particularly preferably in an amount of 0.012%, particularly preferably in an amount of 0.02%.

Medicament formulations according to the invention which are administered as single doses, the metering chamber having a volume of 100 μl, preferably contain the dissolved medicament 1 in an amount of 0.0025-0.025%, particularly preferably in an amount of 0.0025-0.0225%, particularly preferably in an amount of 0.004-0.02%, particularly preferably in an amount of 0.005-0.02%, particularly preferably in an amount of 0.005-0.01%, particularly preferably in an amount of 0.01%.

In another particularly preferred form, medicament formulations according to the invention contain only the active substance 1 as sole component as a pharmacologically active ingredient (active substance) for treating inflammatory or obstructive respiratory diseases.

The percentages given within the scope of the present invention are always percent by weight. If proportions by mass of tiotropium are expressed as percent by weight, the corresponding values for the crystalline tiotropium bromide monohydrate that is preferably used within the scope of the production according to the present invention can be obtained by multiplication by the conversion factor 1.2495 based on the molecular mass of the tiotropium bromide.

The formulations for preparing propellant-gas-containing inhalable aerosols contain cosolvents, according to the invention. Preferably, according to the invention, the formulations contain a mixture of water and ethanol as cosolvents. Preferably, also, the formulations contain an amount of water of 0.5-2.5% and 15-40% ethanol. Particularly preferably the formulations contain a proportion of water of 0.5-2% and 15-40% ethanol. Preferably, moreover, the formulations contain a proportion of water of 1-2% and 15-35% ethanol. More preferably the formulations contain a proportion of water of 1-2% and 20-30% ethanol.

According to the invention these quantities of water are preferably added to the propellant gases or the solutions of the active substance 1 and the cosolvents if the propellant gas, propellant gas mixture or formulation does not contain any other water (free water). Technically, the water may be mixed with the propellant gas even before the medicament formulation is prepared, or first the medicament formulation is prepared with anhydrous propellant gas or propellant gas mixture and then the corresponding amount of water is added.

In some cases, the term medicament formulation is used within the scope of the present invention instead of the term solution formulation. The two terms are to be regarded as equivalent within the scope of the present invention.

The propellant-containing inhalable aerosols or solution formulations according to the invention may also contain other constituents such as surface-active agents (surfactants), adjuvants, antioxidants or flavourings.

The surface-active agents (surfactants) optionally present in the solutions according to the invention are preferably selected from the group consisting of Polysorbate 20, Polysorbate 80, Myvacet 9-45, Myvacet 9-08, isopropyl myristate, oleic acid, propyleneglycol, polyethyleneglycol, Brij, ethyl oleate, glyceryl trioleate, glyceryl monolaurate, glyceryl monooleate, glyceryl monostearate, glyceryl monoricinoleate, cetyl alcohol, oleyl oleate, sterylalcohol, cetylpyridinium chloride, block polymers, natural oil, ethanol and isopropanol.

If the solutions according to the invention contain surfactants these are preferably used in an amount of 0.0005-1%, particularly preferably 0.005-0.5%. In another preferred embodiment the solutions according to the invention do not contain any surface-active agents (surfactants).

The antioxidants optionally contained in the solutions according to the invention are preferably selected from the group consisting of ascorbic acid, citric acid, sodium edetate, editic acid, tocopherols, butylhydroxytoluene, butylhydroxyanisole and ascorbylpalmitate, while tocopherols, butylhydroxytoluene, butylhydroxyanisole or ascorbylpalmitate are preferably used.

The flavourings optionally contained in the solutions according to the invention are preferably selected from the group consisting of peppermint, saccharine, Dentomint®, aspartame and ethereal oils (for example cinnamon, aniseed, menthol, camphor), peppermint or Dentomint® being particularly preferred.

The solutions according to the invention may be prepared using methods known in the art. For this, the constituents of the formulation are mixed with the propellant gas or gases (optionally at low temperatures) and filled into suitable containers.

Surprisingly it has been found that the problem of the invention is solved by the preparation of a medicament for treating inflammatory or obstructive respiratory complaints in the form of a medicament formulation, the medicament formulation containing as active substance exclusively 1 in dissolved form, characterised in that the medicament formulation contains the propellant gas HFA 134a and as other ingredients 0.005-0.03% of the active substance 1, a mixture of the two cosolvents ethanol and water, the ethanol content being between 15-40% and the water content being between 0.5-2.5%, and an acid, which is present in an amount such that the pH is between 6 and 8.

In one particular embodiment the problem of the invention is solved by a medicament formulation for the treatment of inflammatory or obstructive respiratory complaints, the medicament formulation containing as active substance exclusively 1 in dissolved form, and is characterised in that the medicament formulation contains the propellant gas HFA 134a, and as other ingredients 0.005-0.03% of the active substance 1, a mixture of the two cosolvents ethanol and water, the ethanol content being between 15-40% and the water content being between 0.5-2.5%, and an organic acid in a concentration of between 0.005-0.1%, preferably between 0.008-0.09%, more preferably between 0.01-0.08, more preferably between 0.02-0.06, and more preferably between 0.02-0.05%.

In one particular embodiment the problem of the invention is solved by a medicament formulation for the treatment of inflammatory or obstructive respiratory complaints, the medicament formulation containing as active substance exclusively 1 in dissolved form, and is characterised in that the medicament formulation contains the propellant gas HFA 134a, and as other ingredients 0.005-0.03% of the active substance 1, a mixture of the two cosolvents ethanol and water, the ethanol content being between 15-40% and the water content being between 0.5-2.5%, and an inorganic acid in a concentration that corresponds to a quantity of H⁺-ions that can be obtained by the addition of a 1-3% proportion of a 0.01 normal acid, preferably an inorganic acid in a concentration that corresponds to a quantity of H⁺-ions that can be obtained by the addition of a 1.5-2.5% proportion of a 0.01 normal acid.

Corresponding amounts of inorganic acid that correspond to the inventive quantity of H⁺-ions can be obtained by the addition of 1-3% of a 0.01 normal acid or 0.1-0.3% of a 0.1 normal acid or by the addition of a corresponding amount that can be determined by conversion in a comparable manner. Corresponding amounts of inorganic acid that correspond to the quantity of H⁺-ions preferred according to the invention can be obtained by the addition of 1.5-2.5% of a 0.01 normal acid or 0.15-0.25% of a 0.1 normal acid or by the addition of a corresponding amount that can be determined by conversion in a comparable manner.

In one particular embodiment the invention relates to a medicament formulation with the properties mentioned hereinbefore or hereinafter, which is further characterised in that the medicament formulation contains as acid an organic acid, preferably citric acid in a concentration of between 0.005-0.1%, preferably between 0.008-0.09%, more preferably between 0.01-0.08, more preferably between 0.02-0.06, and more preferably between 0.02-0.05%.

In one particular embodiment the invention relates to a medicament formulation with the properties mentioned hereinbefore or hereinafter, which is further characterised in that the medicament formulation contains as acid an organic acid, preferably ascorbic acid in a concentration of between 0.005-0.1%, preferably between 0.008-0.09%, more preferably between 0.01-0.08, more preferably between 0.02-0.06, and more preferably between 0.02-0.05%.

In another particular embodiment the invention relates to a medicament formulation with the properties mentioned hereinbefore or hereinafter, which is further characterised in that the medicament formulation contains as acid an inorganic acid in a concentration that corresponds to a quantity of H⁺-ions that can be obtained by the addition of a 1-3% proportion of a 0.01 normal acid, preferably an inorganic acid in a concentration that corresponds to a quantity of H⁺-ions that can be obtained by the addition of a 1.5-2.5% proportion of a 0.01 normal acid.

In another particular embodiment the invention relates to a medicament formulation with the properties mentioned hereinbefore or hereinafter, which is further characterised in that the medicament formulation contains as acid aqueous hydrochloric acid in a concentration that corresponds to a quantity of H⁺-ions that can be obtained by the addition of a 1-3% proportion of a 0.01 normal acid, preferably an inorganic acid in a concentration that corresponds to a quantity of H⁺-ions that can be obtained by the addition of a 1.5-2.5% proportion of a 0.01 normal acid, preferably in that the medicament formulation contains a 1-3% proportion of a 0.01 molar hydrochloric acid, more preferably in that the medicament formulation contains a 1.5-2.5% proportion of a 0.01 molar hydrochloric acid.

In another particular embodiment the invention relates to a medicament formulation with the properties mentioned hereinbefore or hereinafter, which is further characterised in that the medicament formulation contains as acid aqueous sulphuric acid in a concentration that corresponds to a quantity of H⁺-ions that can be obtained by the addition of a 1-3% proportion of a 0.01 normal acid, preferably an inorganic acid in a concentration that corresponds to a quantity of H⁺-ions that can be obtained by the addition of a 1.5-2.5% proportion of a 0.01 normal acid.

In another particular embodiment the invention relates to a medicament formulation with the properties mentioned hereinbefore or hereinafter, which is further characterised in that the nominal dose of the single dosage is between 5.5 μg and 6.5 μg, the delivered dose is between 4.5-5.6 μg, and the pulmonary dose is between 2-3 μg and the mean aerodynamic particle size is between 2.5-3.5 μm.

In another particular embodiment the invention relates to a medicament formulation with the properties mentioned hereinbefore or hereinafter, which is further characterised in that the nominal dose of the single dosage is between 5.5 μg and 6.5 μg, and the delivered dose is preferably between 4.7-5.4 μg, particularly preferably between 4.8-5.3 μg, more particularly preferably between 4.9-5.2 μg, and most particularly preferably between 5-5.1 μg.

In another particular embodiment the invention relates to a medicament formulation with the properties mentioned hereinbefore or hereinafter, which is further characterised in that the nominal dose of the single dosage is between 5.5 μg and 6.5 μg, and the pulmonary dose is preferably between 2.0-3.0 μg, particularly preferably between 2.1-3.0 μg, most particularly preferably between 2.3-2.9 μg, and even more particularly preferably between 2.4-2.7 μg.

In another particular embodiment the invention relates to a medicament formulation with the properties mentioned hereinbefore or hereinafter, which is further characterised in that the nominal dose of the single dosage is between 5.5 μg and 6.5 μg, and the mean aerodynamic particle size is preferably between 2.6-3.4 μm, more preferably between 2.7-3.3 μm.

In another particular embodiment the invention relates to a medicament formulation with the properties mentioned hereinbefore or hereinafter, which is further characterised in that the dose is administered by the application of a single dose by a spray jet, in which a dose flows out of the metering chamber by actuation of the inhaler and is expelled from the nozzle and the resulting aerosol is characterised in that the nominal dose of the single dosage is between 5.5 μg and 6.5 μg, the delivered dose is between 4.5-5.6 μg, and the pulmonary dose is between 2-3 μg and the mean aerodynamic particle size is between 2.5-3.5 μm.

In another particular embodiment the invention relates to a medicament formulation with the properties mentioned hereinbefore or hereinafter, which is further characterised in that the bore diameter of the spray head used has a diameter of between 0.2-0.3 mm, preferably between 0.20-0.27 mm, most preferably between 0.20 and 0.25 mm, and the aerosol formed is characterised in that the nominal dose of the single dosage is between 5.5 μg and 6.5 μg, the delivered dose is between 4.5-5.6 μg, and the pulmonary dose is between 2-3 μg and the mean aerodynamic particle size is between 2.5-3.5 μm.

In another particular embodiment the invention relates to a medicament formulation with the properties mentioned hereinbefore or hereinafter, which is further characterised in that the metering chamber has a volume of between 25 μl and 100 μl, preferably 50 μl, and the aerosol formed is characterised in that the nominal dose of the single dosage is between 5.5 μg and 6.5 μg, the delivered dose is between 4.5-5.6 μg, and the pulmonary dose is between 2-3 μg and the mean aerodynamic particle size is between 2.5-3.5 μm.

In another particular embodiment the invention relates to a medicament formulation with the properties mentioned hereinbefore or hereinafter, which is further characterised in that the sealing rings in the valve head are made from ethylene-propylene-diene monomer (EPDM), and the aerosol formed is characterised in that the nominal dose of the single dosage is between 5.5 μg and 6.5 μg, the delivered dose is between 4.5-5.6 μg, and the pulmonary dose is between 2-3 μg and the mean aerodynamic particle size is between 2.5-3.5 μm.

In another particular embodiment the invention relates to a medicament formulation with the properties mentioned hereinbefore or hereinafter, which is further characterised in that the active substance is tiotropium bromide.

In another particular embodiment the invention relates to the use of a tiotropium bromide-containing medicament formulation for the treatment of inflammatory or obstructive respiratory complaints, the medicament formulation being characterised by the properties mentioned hereinbefore or hereinafter.

In another particular embodiment the invention relates to the active substance tiotropium 1, which is present in one of the medicament formulations mentioned hereinbefore or hereinafter, for use as a medicament for the treatment of inflammatory or obstructive respiratory complaints, the medicament being administered
once a day,
in a nominal dose of between 5.5 μg and 6.5 μg,
in a delivered dose of between 4.5-5.6 μg, and
in a pulmonary dose of between 2-3 μg
   the mean aerodynamic particle size being between 2.5-3.5 μm.

In another particular embodiment the invention relates to the active substance tiotropium 1, which is present in one of the medicament formulations mentioned hereinbefore or hereinafter, for use as a medicament for the treatment of inflammatory or obstructive respiratory complaints, the medicament being administered
once a day,
in a nominal dose of between 5.5 μg and 6.5 μg,
in a delivered dose of between 4.5-5.6 μg and
in a pulmonary dose of between 2.0-3.0 μg, preferably between 2.1-3.0 μg, particularly preferably between 2.3-2.9 μg, and most particularly preferably between 2.4-2.7 μg
   the mean aerodynamic particle size being between 2.5-3.5 μm.

In another particular embodiment the invention relates to the active substance tiotropium 1, which is present in one of the medicament formulations mentioned hereinbefore or hereinafter, for use as a medicament for the treatment of inflammatory or obstructive respiratory complaints, the medicament being administered
once a day,
in a nominal dose of between 5.5 μg and 6.5 μg,
in a delivered dose of between 4.7-5.4 μg, preferably between 4.8-5.3 μg, particularly preferably between 4.9-5.2 μg, as well as most particularly preferably between 5-5.1 μg and
in a pulmonary dose of between 2-3 μg
   the mean aerodynamic particle size being between 2.5-3.5 μm.

The above-mentioned propellant-gas-containing solutions according to the invention may be administered using inhalers known in the art (pMDIs=pressurized metered dose inhalers). Accordingly, in another aspect, the present invention relates to medicaments in the form of solutions as described hereinbefore combined with one or more inhalers suitable for administering these solutions. Moreover the present invention relates to inhalers, characterised in that they contain the propellant-gas-containing solutions according to the invention as described above. The present invention further relates to containers (e.g. cartridges), which are fitted with a suitable valve that is prepared before use with regard to the water content. The containers may be used in a suitable inhaler and contain one of the above-mentioned propellant-gas-containing solutions according to the invention. Suitable containers (e.g. cartridges) and processes for filling these cartridges with the propellant-gas-containing solutions according to the invention are known from the prior art.

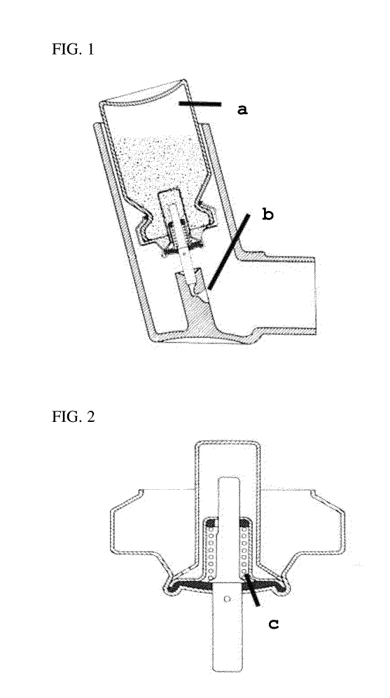

The cartridge (cf. FIG. 1, section “a”) contains the medicament as a solution. The formulation contains, besides the active substance 1, other chemical substances as well, which are important for stability and product performance. Above the liquid level, further gaseous propellant gas is present. The propellant gas in the metering chamber causes the medicament formulation to be expelled explosively when the metered dose aerosol is actuated. (For the purposes of the present invention the word cartridge is equivalent to canister).

In the nozzle (cf. FIG. 1, section “b”) are formed the small droplets of liquid that contain the medicament. The size of these droplets is important in order to obtain good deposition in the lungs. The nozzle is characterised by the bore diameter (narrowest diameter) of the spray head “b” used.

The amount of medicament expelled is metered by the metering chamber (cf. FIG. 2, section “c”).

The droplet size of a metered dose aerosol can be influenced by the nozzle design (bore diameter of the spray head, size of the metering chamber) and by the composition of the solution formulation. In particular, there is the possibility of varying the droplet size by the choice of propellant gas. The smaller the nozzle diameter of the spray head, the smaller the droplets produced—although limits are set on the design, as nozzles that are too small become blocked. The lower limit for the bore is 0.16 mm, as a smaller nozzle can easily become blocked.

The use of a spray head with a bore diameter of between 0.20-0.27 mm is particularly preferred within the scope of the present invention. It is also particularly preferable to use a metering chamber with a volume of between 25 μl and 100 μl, particularly preferably 50 μl. Commercially obtainable mouth tubes (e.g. as manufactured by RPC Formatec GmbH, Germany) may be used for the metered dose aerosols according to the invention.

In another aspect the present invention relates to a process for metering a medicament of a formulation according to the invention in order to produce propellant-gas-containing inhalable aerosols, comprising the steps of
removing the lid
vigorously shaking, with the canister facing upwards
breathing out
placing the metered dose aerosol between the lips
slowly breathing in and immediately . . .
. . . pressing on the canister
breathing in slowly and completely
holding one's breath if possible for 10 seconds

The following Examples serve to illustrate the present invention by way of example, without restricting it to their contents.

### FORMULATION EXAMPLES (COMPOSITION)

1.

| | | |
|---|---|---|
| Tiotropium bromide monohydrate | 0.04% | by weight |
| citric acid | 0.05% | by weight |
| water | 1.0% | by weight |
| ethanol | 30.0% | by weight |
| HFA134a | 68.9% | by weight |
| Total | 100% | by weight (13.5 g) |

2.

| | | |
|---|---|---|
| Tiotropium bromide monohydrate | 0.01% | by weight |
| citric acid | 0.02% | by weight |
| water | 1.5% | by weight |
| ethanol | 15.0% | by weight |
| HFA134a | 83.5% | by weight |
| Total | 100% | by weight (14.5 g) |

3.

| | | |
|---|---|---|
| Tiotropium bromide monohydrate | 0.02% | by weight |
| citric acid | 0.04% | by weight |
| water | 2.0% | by weight |
| ethanol | 35.0% | by weight |
| BHT (butylhydroxytoluene) | 0.1% | by weight |
| HFA134a | 62.8% | by weight |
| Total | 100% | by weight (13.2 g) |

4.

| | | |
|---|---|---|
| Tiotropium bromide monohydrate | 0.02% | by weight |
| sulphuric acid | 0.1% | by weight |
| water | 1.0% | by weight |
| ethanol | 20.0% | by weight |
| HFA134a | 78.9% | by weight |
| Total | 100% | by weight (14.2 g) |

5.

| | | |
|---|---|---|
| Tiotropium bromide monohydrate | 0.04% | by weight |
| hydrochloric acid | 0.2% | by weight |
| water | 0.5% | by weight |
| ethanol | 15.0% | by weight |
| HFA134a | 84.3% | by weight |
| Total | 100% | by weight (14.6 g) |

We claim:

1. An inhaler containing a pharmaceutical aerosol formulation consisting of:
(i) a propellant gas HFA 134a;
(ii) 0.005-0.03% of an active substance tiotropium (1);
(iii) a mixture of co-solvents ethanol and water, the ethanol content being between 15-40% and the water content being between 0.5-2.5%; and
(iv) an acid,
wherein a solution consisting of (ii), (iii), and (iv) has a pH between 6 and 8, and wherein a single actuation from the inhaler has the following delivery characteristics:
(a) a nominal dose between 5.5 μg and 6.5 μg;
(b) a delivered dose between 4.5-5.6 μg;
(c) a pulmonary dose (Fine Particle Dose) between 2-3 μg; and
(d) a mean aerodynamic particle size between 2.5-3.5 μm.

2. An inhaler containing a pharmaceutical aerosol formulation consisting of:
(i) a propellant gas HFA 134a;
(ii) 0.005-0.03% of an active substance tiotropium (1);
(iii) a mixture of co-solvents ethanol and water, the ethanol content being between 15-40% and the water content being between 0.5-2.5%; and
(iv) an acid,
wherein a solution consisting of (ii), (iii), and (iv) has a pH between 6 and 8, and wherein an inhaled dose is administered to a patient from a spray jet where the dose flows out of the metering chamber by actuation of the inhaler to expel it from a nozzle, an aerosol thus formed having the following delivery characteristics:
(a) a nominal dose between 5.5 μg and 6.5 μg;
(b) a delivered dose between 4.5-5.6 μg;
(c) a pulmonary dose (Fine Particle Dose) between 2-3 μg; and
(d) a mean aerodynamic particle size between 2.5-3.5 μm.

3. The inhaler according to claim 2, wherein a bore diameter of a spray head of the spray jet has a diameter of between 0.2-0.3 mm.

4. The inhaler according to claim 2, wherein the metering chamber has a volume of between 25 μl and 100 μl.

5. The inhaler according to claim 3, wherein the metering chamber has a volume of between 25 μl and 100 μl.

6. The inhaler according to claim 2, wherein the metering chamber has a volume of 50 μl.

7. The inhaler according to claim 3, wherein the metering chamber has a volume of 50 μl.

8. The inhaler according to claim 4, wherein sealing rings in a valve head are ethylene-propylene-diene monomer (EPDM).

9. A method for treating inflammatory or obstructive respiratory complaints in a patient, the method comprising administering to the patient the active substance tiotropium (1) with the inhaler according to claim 1.

10. A method for treating inflammatory or obstructive respiratory complaints in a patient, the method comprising administering to the patient the active substance tiotropium (1) with the inhaler according to claim 2.

11. The inhaler according to claim 1, wherein the active substance tiotropium (1) is tiotropium bromide.

12. The inhaler according to claim 1, wherein the acid of the pharmaceutical aerosol formulation is an organic acid.

13. The inhaler according to claim 12, wherein the organic acid of the pharmaceutical aerosol formulation is citric acid in a concentration of between 0.005-0.1%.

14. The inhaler according to claim 1, wherein the acid of the pharmaceutical aerosol formulation is an inorganic acid in a concentration that corresponds to a quantity of H⁺ ions that can be obtained by the addition of a 1-3% proportion of a 0.01 normal acid.

15. The inhaler according to claim 14, wherein the inorganic acid of the pharmaceutical aerosol formulation is aqueous hydrochloric acid in a concentration that corresponds to a quantity of H⁺ ions that can be obtained by the addition of a 1-3% proportion of a 0.01 normal acid.

16. The inhaler according to claim 14, wherein the inorganic acid of the pharmaceutical aerosol formulation is aqueous sulfuric acid in a concentration that corresponds to a quantity of H⁺ ions that can be obtained by the addition of a 1-3% proportion of a 0.01 normal acid.
